(19) United States
(12) Patent Application Publication
(10) Pub. No.: US 2023/0165998 A1
(43) Pub. Date: Jun. 1, 2023

(54) DEVICE AND METHOD FOR PURIFYING A VEHICLE CABIN

(71) Applicant: RGF ENVIRONMENTAL GROUP, INC., Riviera Beach, FL (US)

(72) Inventors: Walter B. ELLIS, Jupiter, FL (US); Sergei CHTCHAVELEV, Greenacres, FL (US)

(73) Assignee: RGF ENVIRONMENTAL GROUP, INC., Riviera Beach, FL (US)

(21) Appl. No.: 17/993,692

(22) Filed: Nov. 23, 2022

Related U.S. Application Data
(60) Provisional application No. 63/283,791, filed on Nov. 29, 2021.

(51) Int. Cl. A61L 9/20; B60H 3/00

(52) U.S. Cl. CPC A61L 9/205; B60H 3/0078; A61L 2209/111; A61L 2209/16

(57) ABSTRACT

A device and method for purifying a vehicle cabin is provided. The device comprises a housing, a plurality of light emitting diode (LED) modules each containing an LED, wherein the LED modules are positioned at least partially within the housing, a catalytic target structure, wherein the structure is located below at least one of the LED modules in the plurality of LED modules, a plurality of reflectors, wherein the reflectors are located below at least one of the LED modules in the plurality of LED modules, a plurality of fans, wherein the fans are located at least partially within the housing, a plurality of photocatalyst filters positioned at least partially within the housing, wherein at least one of the plurality of photocatalyst filters is in parallel with at least one of the LED modules in the plurality of LED modules, and a control unit located at least partially within the housing, wherein the control unit is operatively connected to the plurality of LED modules.

### CROSS REFERENCE TO RELATED APPLICATION(S)

This application claims the benefit of U.S. provisional application No. 63/283,791, filed Nov. 29, 2021, which is incorporated by reference herein in its entirety.

### TECHNICAL FIELD

Disclosed are embodiments related to a device and method for purifying a vehicle cabin and, more specifically, to a device and method for purifying a vehicle cabin using ultraviolet LEDs and catalytic target structures configured in an arrangement that generates advanced oxidation products that react with and neutralize compounds in the air and on surfaces in the vehicle cabin, including microbes, such as bacteria, viruses and mold, odor causing chemicals, and other organic and inorganic chemicals.

### BACKGROUND

Germicidal ultraviolet light rays have been used for inactivating microorganisms such as viruses and bacteria. Germicidal ultraviolet light, however, is effective in reducing only the airborne microorganisms that pass directly through the light rays, and has little to no effect on gasses, vapors, or odors.

Alternatively, advanced oxidation processes may be used to eliminate microorganisms, as well as gasses, vapors, and odors. In an advanced oxidation process, advanced oxidation products (“AOPs”) are produced, and subsequently destroy and/or inactivate undesired compounds in the environment. The production of AOPs may be catalyzed by ultraviolet light.

Commonly-owned U.S. Pat. No. 7,988,923, incorporated herein by reference in its entirety and included in Appendix A, describes a device, system, and method for using UV light to generate advanced oxidation products (“AOPs”) in an advanced oxidation process. In this system, a light source producing multiple wavelengths of UV light is provided adjacent to a catalytic surface of a catalytic target structure. The catalytic surface is coated with a thin coating comprising hydrophilic material, thus promoting hydration of the catalytic surface from ambient moisture. AOPs are formed when the UV light reacts with the hydrate on the photocatalytic surfaces, and also within the air itself between the catalytic target structure, AOPs and the UV source. Additionally, any trace amounts of ozone created within the system go through ozone photodecomposition reactions within the cell, resulting in additional in air production of a variety of AOPs. The entirety of the AOPs produced by this system may then be used to eliminate gasses, vapors, odors, and/or microbes in the environment, while also eliminating ozone release from the system (to non, or near non detectable levels) Hydrogen Peroxide is one of the specific AOP's targeted for production via this system.

Light emitting diodes (LEDs) are efficient devices for applying UV light, including the wavelengths of UV light that may be used to purify an environment. UV LEDs can, however, discharge significant heat which, if not dissipated, can interfere with the operation of the UV LED. Moreover, the significant heat generated by the LEDs and the cycling of such LEDs on and off may result in significant expansion and contraction of the structure configured to house the LEDs. Moreover, LEDs have relatively low tolerance for humidity and may fail if exposed to a humid environment for an extended period of time.

Commonly-owned U.S. Pat. No. 11,032,887, incorporated herein by reference in its entirety and included in Appendix A, describes systems and methods for applying ultraviolet (UV) light to an environment, which may include an elongate first body having a first side wall, a second side wall opposite the first side wall, and a bottom wall. The first body may define a lengthwise channel between the first side wall and the second side wall. The first body may have a first groove disposed along an inner surface of the first side wall, a second groove disposed along an inner surface of the second side wall, and a cover which may be coupled to the first body via the first groove and the second groove. The first body and the cover may collectively enclose at least a portion of the channel. The system may include an LED disposed within the channel. The system may also include a processor and plurality of LED arrays. Each LED array may include one or more LEDS which may be powered on and off together. The system may be configured to a apply a pulsed power input to the first LED array during a first timeslot, apply the pulsed power input to a second LED array during a second timeslot, and, if the plurality of LED arrays includes more than two LED arrays, apply the pulsed power input to each remaining LED array in respective timeslots. These steps may be performed such that power is applied to only one LED array of the plurality of LED arrays at any given time.

This above-described control system and method offers several distinct advantages. By pulsing the individual LED arrays, it is possible to turn on and off the LEDs as desired. The longer the LED is off, the less heat is generated and the lower the junction temperature of the LED will be. This lower temperature significantly increases the working life of the LEDs in the system. Further, by pulsing each LED on and then off, it is possible to drive each of the LED's at a higher current, providing more delivered germicidal UV output energy, while keeping a lower junction temperature as compared to an equivalently current driven non-pulsed LED. This allows the germicidal efficacy of the system to be improved at the same time as the LED life is increased. Relative to an LED that is not pulsed with equivalent average power consumption, a pulsed LED may have higher peak power output. Pulsed LEDs are found to provide improved germicidal and anti-microbial activity relative to LEDs that are not pulsed.

Moreover, because heat dissipation is improved, it is possible to use smaller heatsinks for a given array, thereby reducing manufacturing costs. Additionally, for a given heatsink arrangement, the LED arrays can be used in hotter operating environments than would otherwise be possible.

Total power consumption can also be reduced. By pulsing each LED array in the system at a different time, the total current for the full system may be proportionally reduced by the number of actual individual LED arrays in the system. Considering a system with 10 LED arrays, for example, in which each array draws 1 amp of current, the current required for a full non-pulsed array would be 10 amps. By utilizing the method described above in which power is supplied to each LED array in sequence, the power requirement for the full system drops to just 1 amp. This allows circuit elements (e.g., integrated circuit components, traces, wire gauges, connectors, etc.) that are shared between the LED arrays to be sized for just one amp, as opposed to 10 amps. In the above example, a wire trace may be sized such that it can safely carry 1 amp, but would fail under a current of 10 amps. This may significantly reduce both component costing and the overall packaging size of all the components needed (making the final product less expensive and smaller). This may also require less input current from the system in which the system is installed, since only one of the LED arrays may draw current at any given time. In applications where having more efficient UV output and longer UV LED life are not a concern, non-pulsed circuits may also be used.

There exists a need in the art, however, to provide cost-effective devices and methods for a significantly improved oxidation process that promotes high efficiency formation AOPs to react with and neutralize compounds, including microbes, such as bacteria, viruses and mold, odor causing chemicals, and other organic and inorganic chemicals, in the air and on surfaces in a vehicle cabin, while, at the same time, housing LED lights that are capable of dissipating the heat generated by the LEDs and have sufficient mechanical and chemical durability to withstand the constant temperature fluctuations, while simultaneously protecting the LEDs.

Moreover, a need exists to provide such devices and methods using ultraviolet LEDs and catalytic target structures that generate advanced oxidation products (“AOPs”) in an advanced oxidation process to purify a vehicle cabin that are zero ozone and non-ionizing.

### SUMMARY

The following description presents a simplified summary in order to provide a basic understanding of some aspects described herein. This summary is not an extensive overview of the claimed subject matter. It is intended to neither identify key or critical elements of the claimed subject matter nor delineate the scope thereof.

According to a first aspect, a device for purifying a vehicle cabin is provided. The device includes a housing and a plurality of light emitting diode (LED) modules each containing an LED, wherein the LED modules are positioned at least partially within the housing\. The device further includes a catalytic target structure, wherein the structure is located below at least one of the LED modules in the plurality of LED modules. the device further includes a plurality of reflectors, wherein the reflectors are located below at least one of the LED modules in the plurality of LED modules. the device further includes a plurality of fans, wherein the fans are located at least partially within the housing. The device further includes a plurality of photocatalyst filters positioned at least partially within the housing, wherein at least one of the plurality of photocatalyst filters is in parallel with at least one of the LED modules in the plurality of LED modules. the device further includes a control unit located at least partially within the housing, wherein the control unit is operatively connected to the plurality of LED modules.

In some embodiments, the plurality of LED modules includes a first LED module positioned at least partially within the housing, wherein the first LED module emits ultraviolet light at a first wavelength, a second LED module positioned at least partially within the housing, wherein the second LED module emits ultraviolet light at a second wavelength, and a third LED module positioned at least partially within the housing, wherein the third LED module emits ultraviolet light at a third wavelength.

In some embodiments, the first LED module and the third LED module emit ultraviolet light at a wavelength between 300 and 400 nm and the second LED module emits ultraviolet light at a wavelength between 200 and 300 nm. In some embodiments, the first LED module and the third LED module emit ultraviolet light at a wavelength of 365 nm and the second LED module emits ultraviolet light at a wavelength of 265-275 nm.

In some embodiments, the catalytic target structure is located below the second LED module. In some embodiments, the plurality of reflectors includes a first reflector located above the second LED module and a second reflector located below the second LED module. In some embodiments, at least one of the plurality of reflectors is a flat surface comprising a highly UV reflective material.

In some embodiments, the reflective material is selected from a group consisting of aluminum, aluminum foil, stainless steel, and polytetrafluoroethylene. In some embodiments, the first reflector is located above the second LED module, the catalytic target structure is located below the second LED module and the second reflector is located below the catalytic target structure.

In some embodiments, the plurality of photocatalyst filters comprises a first photocatalyst filter and a second photocatalyst filter, wherein the first LED module is in parallel with the first photocatalyst filter and the second LED module is in parallel with the second photocatalyst filter. In some embodiments, the control unit is configured to control at least one of the plurality of LED modules. the fan series, and the catalytic target structure.

In some embodiments, the control unit is configured to control ambient conditions within the housing. In some embodiments, the ambient conditions are selected from a group consisting of humidity, temperature, selective gases, noise level, and air quality. In some embodiments, the plurality of fans are positioned in a series. In some embodiments, the device emits zero to near zero ozone. In some embodiments, the device is configured such that the device generates 10-70 parts per billion of ROS compounds in the vehicle cabin the device is purifying.

In some embodiments, at least one of the plurality of photocatalyst filters is in a honeycomb configuration. In some embodiments, at least one of the plurality of photocatalyst filters is composed of a material selected from a group consisting of aluminum oxide, silicon dioxide, magnesium oxide, and titanium oxide.

According to a second aspect, a method for purifying a vehicle cabin is provided. The method includes supplying an air product. The method further includes receiving the air product within a purification device. The method further includes processing the air product within the purification device by means of a photocatalytic configuration which initiates a chemical reaction utilizing airborne oxygen and water producing a plurality of reactive oxygen species, wherein the reactive oxygen species chemically react with gases, particles, and surface contaminants within the vehicle cabin. The method further includes outputting the processed air product into a vehicle cabin.

According to a third aspect, a system for purifying a vehicle cabin is provided. The system includes an air supply that supplies an air product. The system further includes a purification device configured to receive the air product and output processed air, the device comprising a housing, a plurality of light emitting diode (LED) modules each containing an LED, wherein the LED modules are positioned at least partially within the housing, a catalytic target structure, wherein the structure is located below at least one of the LED modules in the plurality of LED modules, a plurality of reflectors, wherein the reflectors are located below at least one of the LED modules in the plurality of LED modules, a plurality of fans, wherein the fans are located at least partially within the housing, a plurality of photocatalyst filters positioned at least partially within the housing, wherein at least one of the plurality of photocatalyst filters is in parallel with at least one of the LED modules in the plurality of LED modules, and a control unit located at least partially within the housing, wherein the control unit is operatively connected to the plurality of LED modules, and a vehicle cabin that receives the processed air output from the purification device.

Further variations encompassed within the devices and methods are described in the detailed description of the invention below.

### BRIEF DESCRIPTION OF THE DRAWINGS

The accompanying drawings, which are incorporated herein and form part of the specification, illustrate various embodiments.

FIG. 1 illustrates a first perspective, cross-sectional view of a purification device according to some embodiments

FIG. 2 illustrates a second perspective, cross-sectional view of a purification device according to some embodiments.

FIG. 3 illustrates a first circuit board of a purification device according to some embodiments.

FIG. 4 illustrates a lower case shell of a purification device according to some embodiments.

FIG. 5 illustrates an upper case shell of a purification device according to some embodiments.

FIG. 6 illustrates a lower case door of a purification device according to some embodiments.

FIG. 7 illustrates an LED case bottom cover of a purification device according to some embodiments.

FIG. 8 illustrates an LED case top cover of a purification device according to some embodiments.

FIG. 9 illustrates a cross-section of a fan series of a purification device according to some embodiments.

FIG. 10 illustrates a first view of an LED strip module of a purification device according to some embodiments.

FIG. 11 illustrates a second view of an LED strip module of a purification device according to some embodiments.

FIG. 12 illustrates a third view of an LED strip module of a purification device according to some embodiments.

FIG. 13 illustrates a fourth view of an LED strip module of a purification device according to some embodiments.

FIG. 14 illustrates a catalyst of a purification device according to some embodiments.

FIG. 15 illustrates a reflector of a purification device according to some embodiments.

FIG. 16 illustrates a catalytic target structure in the shape of a grill for a purification device according to some embodiments.

FIG. 17 illustrates various locations where a purification device could be installed in a vehicle cabin according to some embodiments.

FIG. 18 illustrates a purification device for installation in a vehicle cabin cup holder according to some embodiments.

FIG. 19 illustrates a purification device for installation in a vehicle cabin air duct according to some embodiments.

FIG. 20 illustrates a purification device for installation in a vehicle cabin vent according to some embodiments.

FIG. 21 illustrates a purification device for installation in an interior space within a vehicle cabin according to some embodiments.

FIG. 22 is a flow chart illustrating a method for purifying a vehicle cabin according to some embodiments.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

Unless defined otherwise, all terms of art, notations and other technical terms or terminology used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

Unless otherwise indicated or the context suggests otherwise, as used herein, “a” or “an” means “at least one” or “one or more.”

This description may use relative spatial and/or orientation terms in describing the position and/or orientation of a component, apparatus, location, feature, or a portion thereof. Unless specifically stated, or otherwise dictated by the context of the description, such terms, including, without limitation, top, bottom, above, below, under, on top of, upper, lower, left of, right of, in front of, behind, next to, adjacent, between, horizontal, vertical, diagonal, longitudinal, transverse, radial, axial, etc., are used for convenience in referring to such component, apparatus, location, feature, or a portion thereof in the drawings and are not intended to be limiting.

Furthermore, unless otherwise stated, any specific dimensions mentioned in this description are merely representative of an exemplary implementation of a device embodying aspects of the disclosure and are not intended to be limiting.

As used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the embodiments described herein.

Embodiments of the devices and methods for purifying environments disclosed herein can be implemented and used within any vehicle and disposed, for example, in a vehicle's air conditioning system, in a pillar within the vehicle cabin, under a seat in the vehicle cabin, or within any available space within the vehicle cabin. Moreover, while exemplary embodiments are described with reference to an automobile, it should be understood that the devices and methods disclosed herein may be beneficial and applicable to other types of vehicles, including trucks, buses, railed vehicles (trains, trams), watercraft (ships, boats), amphibious vehicles (screw-propelled vehicle, hovercraft), aircraft (airplanes, helicopters, aerostat) and spacecraft.

Embodiments of the devices and methods for purifying environments disclosed herein can be implemented and controlled either by the vehicles integrated control circuits, thereby allowing selective control of the device during any conceivable control mode, or their inputs and outputs, using either standard installed or available installed vehicle sensors, e.g., computer system control modules, air quality sensors, (including but not limited to temperature, humidity, particle, O2, CO2, and CO gas sensors), and the like. Or standalone control modes controlled by either semi-automatic (e.g., vehicle occupancy sensors, window position sensors), or completely manually by electric control circuits operated by standalone in cabin manually activated switches.

Embodiments of the devices and methods for purifying environments disclosed herein can use both integrated fans, of any type suitable for the designated install location and condition (axial, linear, AC/DC, PWM controlled, etc.). Either controlled by the vehicle's computer or any secondary control and input circuits. Or, in other embodiments, have no integrated fans. Whereby the unit is installed within a vehicle's modified or unmodified existing HVAC duct system (in dash, under floor, in ceiling, etc.) and uses the fans of the HVAC system to also move air through the AOP air purifying device to be treated and then dispersed into the cabin.

Any two or more embodiments described in this disclosure may be combined in any way with each other. Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

The purification devices and methods disclosed herein may be used for purifying a vehicle cabin using ultraviolet LEDs and catalytic target structures configured in an arrangement that generates advanced oxidation products that react with and neutralize compounds in the air and on surfaces in the vehicle cabin, including microbes, such as bacteria, viruses and mold, odor causing chemicals, and other organic and inorganic chemicals.

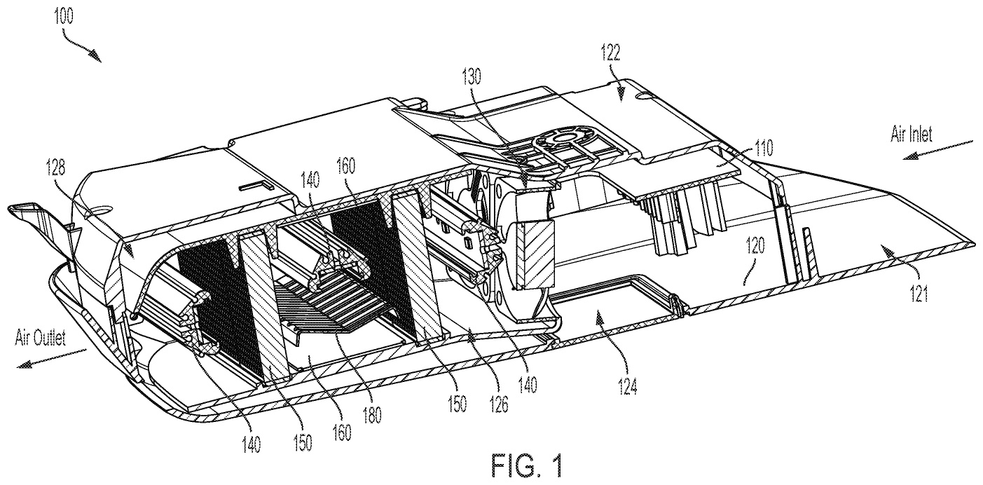

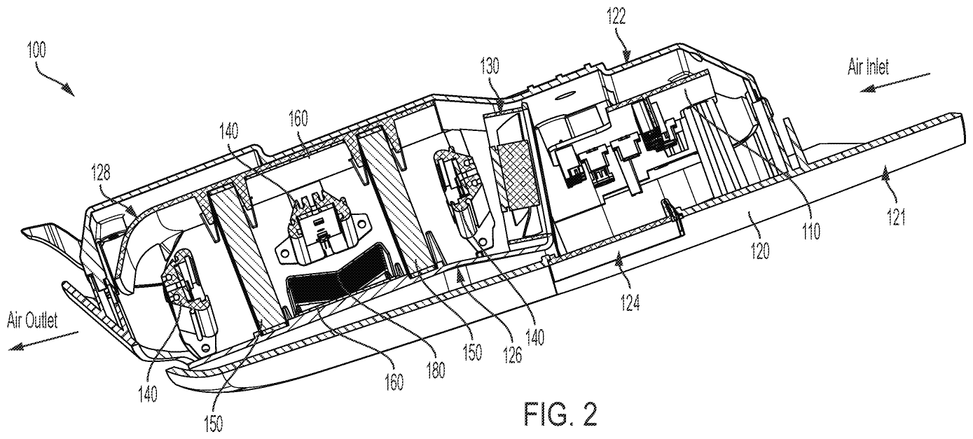

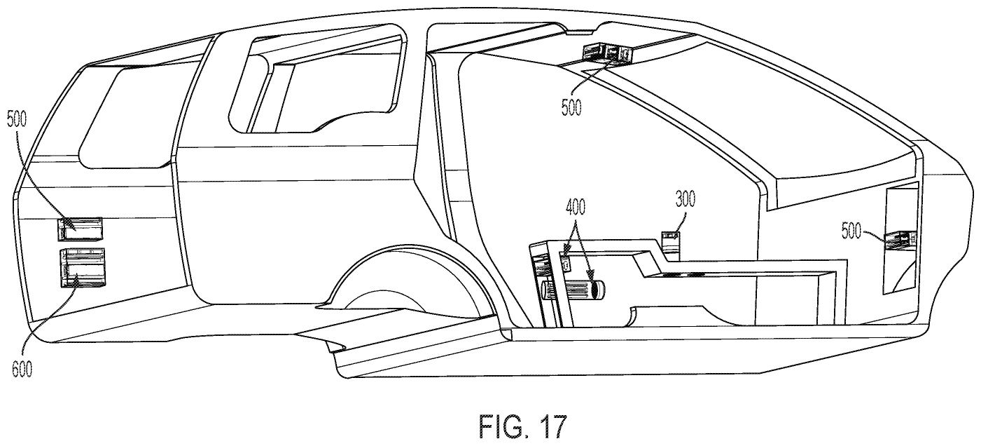

FIG. 1 illustrates an exemplary device 100 for purifying an environment and, more specifically, a vehicle cabin. As shown in FIGS. 1 and 2, air may inlet through one side of the device 100 and exit through the other side of the device 100. In at least this way, the device 100 may purify the air in an environment, such as a vehicle cabin. It will be understood that the device 100 may be installed in a variety of locations in a vehicle 200 (see, e.g., FIG. 17), including in a vehicle's air conditioning system, in a pillar within the vehicle cabin, under a seat in the vehicle cabin, or within any available space within the vehicle cabin. The device 100 may use integrated fans 120 sized per the installed location, or utilize the existing vehicle HVAC fans and ducting as the motive force to move and disperse the treated air.

In some embodiments, the device 100 may include a housing 120. In some embodiments, the housing 120 may have several surfaces, including a lower housing shell 121, an upper housing shell 122, a lower housing door 124, an LED housing bottom 126, and an LED housing top 128. It will be understood that the various components of the housing 120 may fit together in several ways. For example, in some embodiments the components of the housing 120 may be snap-fit together.

In some embodiments, the housing 120 may house one, some, or all of the components of the device 100 described herein. It will be understood that the components of the device 100 may fit into the housing 120 in a variety of ways. For example, and without being limiting, the components of the device 100 may be snap-fit into the housing 120. In some embodiments, the components of the device 100 may be fixed to the housing 120 by means of, for example, screws.

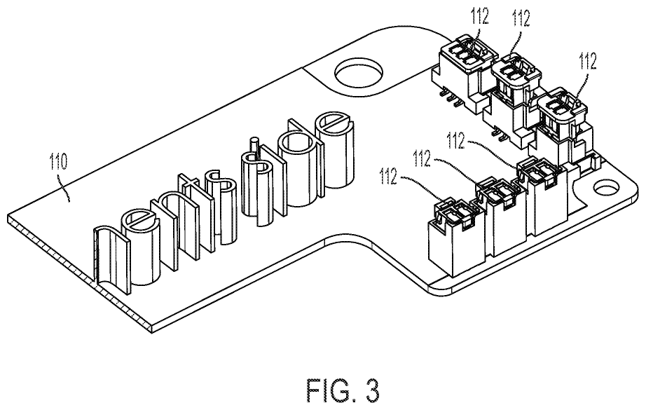

In some embodiments, the device 100 may include a circuit board 110. The circuit board 110 in some embodiments may operate as a control unit for the device 100. As shown in FIG. 3, the circuit board 110 may include a plurality of inputs 112. The circuit board 110 may have fixed electrical components soldered to it, as well as provide electrical connections and controlled open circuits. In some embodiments, the circuit board 110 may be operatively connected to various components of the system 100. For example, the circuit board 110 may be operatively connected to the fan series 130 and control, for example, the speed of the fan series 130. By way of further example, the circuit board 110 may be operatively connected to plurality of LED modules 140.

It will be understood by those of ordinary skill in the art that multiple components of the device 100 may be powered by the circuit board 110 simultaneously. It will be further understood by those of ordinary skill in the art that the inputs 112 of the circuit board 110 may vary according to the needs of the device 100.

In some embodiments, the circuit board 110 may also control various aspects of the device 100. For example, the circuit board 110 may have control features that turn off the system 100 as a response to a high humidity environment or to high temperature. It will be understood that the circuit board 110 may control a variety of ambient conditions within the system 100 by means of controlling, for example, the plurality of fans 130 and/or the LED modules 140. In at least this way, it will be understood, the circuit board 110 may control ambient conditions such as temperature, humidity, noise level, and air quality in the device 100.

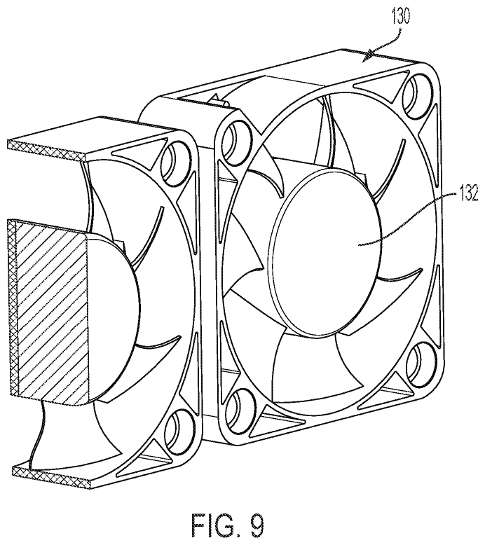

In some embodiments, the device 100 may include a fan series 130. As shown in FIG. 9, the fan series 130 may include a plurality of fans 132. The fans 132 may in some embodiments include a plurality of 12 volt, quiet, long-life fans. As previously mentioned, in some embodiments, the fan series 130 may be operatively connected to the circuit board 110.

In some embodiments, as will be understood by those skilled in the art that the plurality of fans 132 may vary in number based on the design of the system 100. For example, in some embodiments, a greater number of fans 132 may be used for increased air flow and cooling through the device 100. It will be understood that the number of fans 132 may be constrained by the size of the device 100.

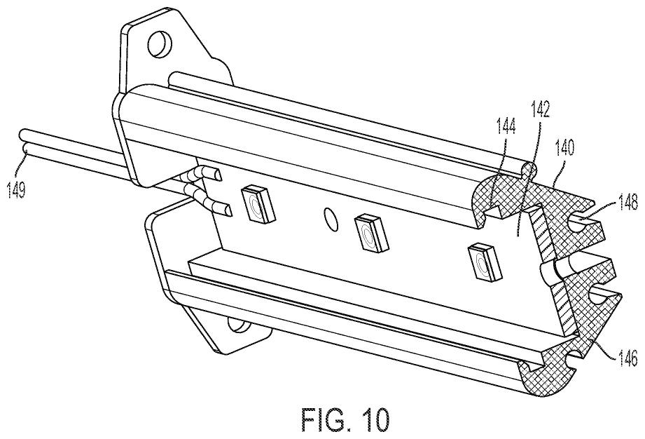

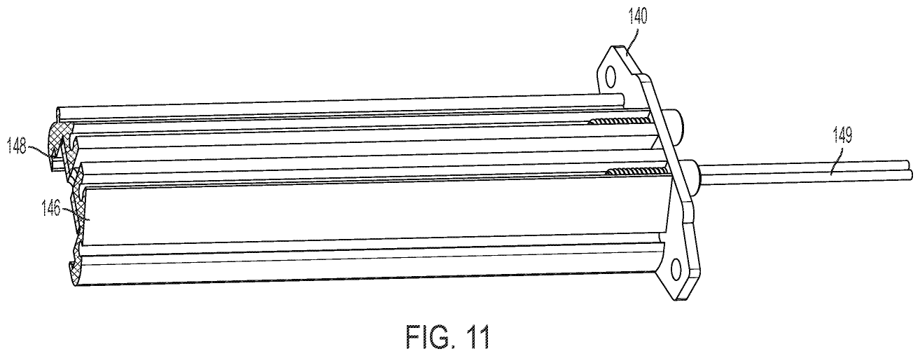

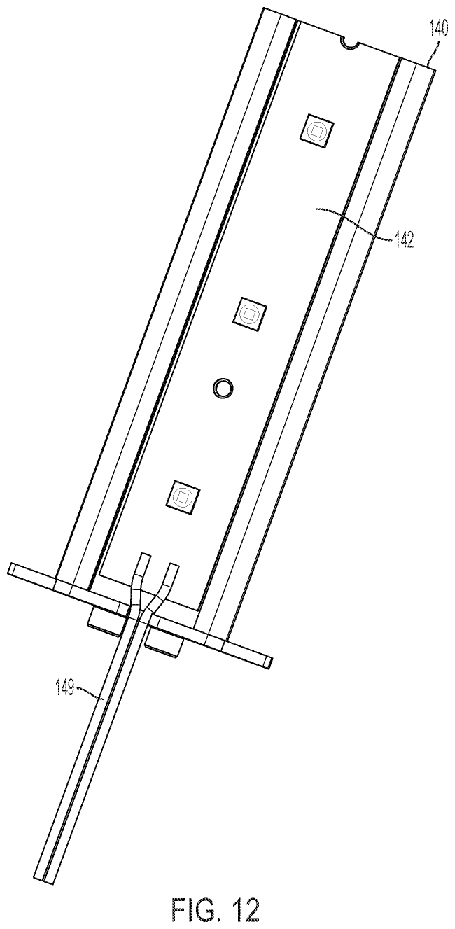

In some embodiments, the device 100 may include a plurality of light emitting diode (LED) modules 140 as shown in FIGS. 1-2 and 10-13. In some embodiments, as shown in FIGS. 10, 12, and 13, the LED modules 140 include an LED 142. The LED 140 may be adapted to emit ultraviolet (UV) light. In some embodiments, the LED 140 may be adapted to emit UV light having a wavelength of 10-400 nm, 100-400 nm, 200-400 nm, 300-400 nm, 200-300 nm, approximately 365 nm, or approximately 265 to 275 nm. It will be understood that this range of frequencies is not exhaustive and the LEDs 140 may be configured to emit UV light at a wide range of wavelengths.

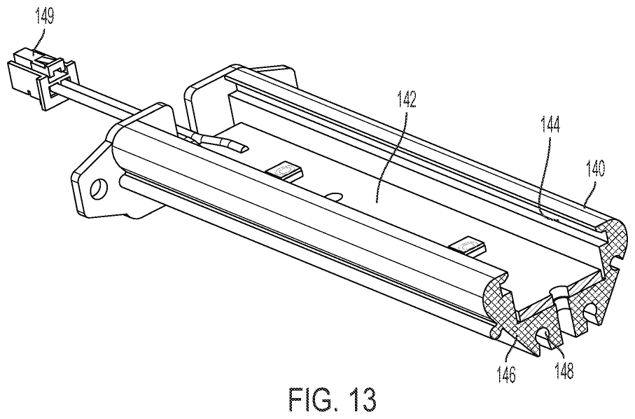

In some embodiments, as shown in FIGS. 10 and 13, the LED modules 140 may have a channel 144.

In some embodiments, as shown in FIGS. 10, 11, and 13, the LED modules 140 may include a heat vent 146 on one side of the module 140. The heat vent 146 may include recesses 148 that reduce the material thickness of the module 140. In some embodiments, the vent 146 increase the surface area of the body through which heat generated by the LED 142 may be dissipated. The recesses 148 may extend along the length of the module 140.

In some embodiments, as shown in FIGS. 10-13, the LED modules 140 may include a connector 149 that may connect to the inputs 112 of the circuit board 110. In at least this way, the circuit board 110 may be operatively connected to and control the LED modules 140.

It will be understood that the device 100 may use a variety of different wavelengths for its LED modules 140, and that the wavelengths may vary between modules 140. It will be understood that the use of different wavelengths of UV light by different LED modules 140 in the system 100 will lead to greater efficiencies of purification for the device 100.

In some embodiments, two LED modules 140 may have LEDs emitting a wavelength of 365 nm and a third LED module 140 may have LEDs emitting a wavelength of 275 or 265 nm. Considering FIGS. 1 and 2, in some embodiments, with air in-letting on the right side of the device 100 and air exiting on the left side of the device 100, the first LED module 140 may emit a wavelength of 365 nm, the second LED module 140 may emit a wavelength of 265-275 nm, and the third LED module 140 may emit a wavelength of 265-275 nm. It will be understood by those of ordinary skill in the art that the wavelengths emitted by the LED modules 140 are not limited to these wavelengths.

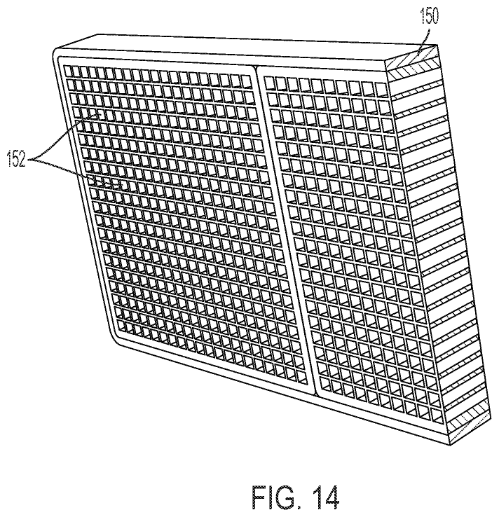

In some embodiments, the device 100 may include a series of photocatalyst filters 150. In some embodiments, the filters 150 may be ceramic. The filters 150 may have a “honeycomb” design with square holes 152 in the filter 150, as shown in FIG. 14. In some embodiments, the filters 150 may be rectangular.

In some embodiments, the photocatalyst filters 150 may be composed of aluminum oxide, silicon dioxide, magnesium oxide, and titanium oxide. In some embodiments, the filters 150 may be 40-50% aluminum oxide, 35-45% silicon dioxide, 2-9% magnesium dioxide, and 10-15% titanium dioxide. It will be understood that the filters 150 may be composed of a variety of materials, however.

In some embodiments, the filters 150 may be free of chemicals and toxins, and the filters 150 may not rely on short-lasting filters (such as activated carbons). In some embodiments, the filters 150 may: have high removal efficiency for volatile organic compounds, be designed for stable immobilizing of titanium oxide, may be reusable by dipping in boiling water, may be free of toxic residue, and may be free of restrictive hazardous substances.

In some embodiments, the photocatalyst filters 150 may be a commercially available product, such as the T1 Photocatalyst Filter produced by Seoul Viosys Co., Ltd. However, it will be understood by those of ordinary skill in the art that there a variety of commercially available photocatalyst filters that may be used.

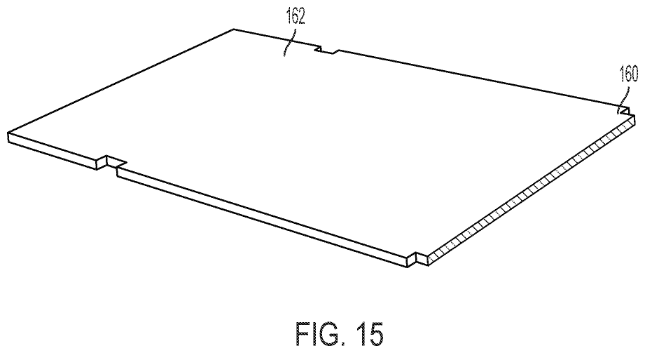

In some embodiments, the device 100 may include a plurality of reflectors 160. As shown in FIGS. 1 and 2, in some embodiments, the reflectors may be in parallel with an LED module 140 and the PHI grill 180. As shown in FIG. 15, in some embodiments, a reflector may be a flat surface with a reflective surface 162.

In some embodiments, the reflectors 160 reflect ultraviolet light to assisted in purifying the environment and improving purification efficiencies. It will be further understood that the reflectors 160 may be configured in a variety of different geometries such that the reflectors 160 optimally distribute ultraviolet light throughout the device 100. The use of a plurality of reflectors 160 as shown in FIGS. 1 and 2 further enhance the efficiencies of the device 100, enabling more ultraviolet light to be reflected within the device 100 to purify the air passing through the device 100. In some embodiments, the reflectors 160 of the device 100 may reflect up to 90% of UV light wavelengths.

In some embodiments, the reflective surface 162 receives ultraviolet light from the LED module 140. In some embodiments, the reflective surface 162 may be composed of a variety of materials, including but not limited to aluminum, aluminum foil, stainless steel, and polytetrafluoroethylene. It will be understood that the reflective surface 162 may be composed of a mixture of materials in some embodiments.

In some embodiments, the device 100 may include a catalytic target structure 180. In some embodiments, and as shown in FIGS. 1, 2, and 16, the target structure 180 may take the form of a grill 180.

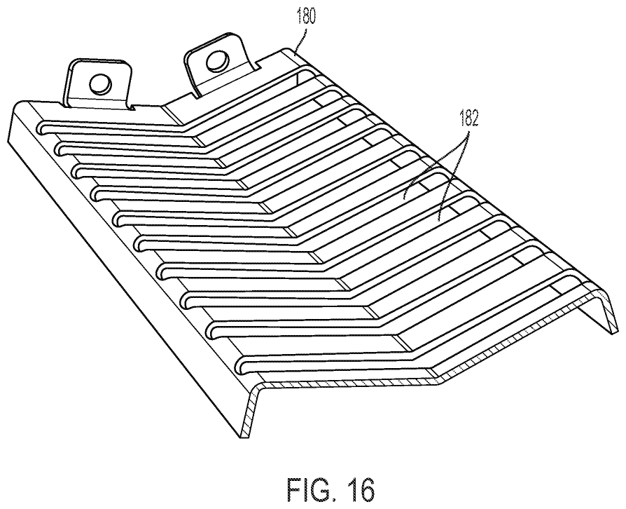

In some embodiments, the structure 180 is also a hydrophilic structure that absorbs water molecules. In some embodiments, as shown in FIG. 16, the structure 180 includes holes or gaps 182 in the structure 180 that allow the passage of gases such as air flowing through the device 100. It will be understood that the structure 180 can be shaped to allow for maximum surface area for receiving the ultraviolet light from the LED modules 140.

In some embodiments, the structure is approximately 50% active catalytic surface with the remaining area being open area, such as the holes 182, to allow the ultraviolet light to pass through the target structure 180. It will be understood that, depending on the requirements of the system 100, the target structure 180 can vary from 0% open area (holes 182) to 95% open area (holes 182).

In some embodiments, the LED modules 140 may be parallel to the structure 180 as shown in FIGS. 1 and 2. The structure 180 may also be located below the LED module 140. It will be understood that the catalytic target structure in some embodiments may conform to the overall shape of the LED module 140 to allow for maximum catalytic target 180 exposure to the ultraviolet light from the LED module 140. However, it will be further understood that, in some embodiments, the structure 180 may be positioned differently in relation to the LED module 140, depending on the requirements of the device 100.

In some embodiments, the catalytic target structure 180 may be composed of a plurality of compounds particularly at the surface of the catalytic target structure 110. Preferably the catalytic target structure 180 may be composed of five compounds: four metallic compounds and a hydrating agent. These compounds preferably include titanium dioxide (TiO2), copper metal (Cu), silver metal (Ag), Rhodium (Rh), and a hydrating agent (such as Silica Gel (tetraalkoxysilanes TMOS, tetramethoxysilane, tetraethoxysilane TEOS)). The hydrating agent may also comprise any suitable compound or combination of compounds that have an affinity to attract or absorb ambient water (i.e., a hydrophilic and hydrating agent).

Some embodiments may use super hydrophilic compounds integrated with TiO2. The catalytic target structure 180 may comprise a base material including a hydrophilic material, a catalytic material, and a ceramic matrix. The base material may be full of tiny channels and connected pores equating to a huge internal surface area, in excess of 750 m² per gram. The higher the porosity of the base material, the more effective the hydraulic attraction (water absorption), and the more surface area available for photocatalytic reactions to occur.

The catalytic target structure 180 may be provided in several different forms configured, for example, to contain hydrophilic granules. The granules may have a diameter in the range of 0.05 mm to 2.5 mm, or a diameter that is greater than or equal to than 2.5 mm.

The hydrophilic material of the catalytic target structure 180 may be formulated to have the unique ability to absorb high quantities of water vapor (i.e. to be extremely hydrophilic). Notably, the hydrophilic material is formulated to also re-release the vast majority of this absorbed water back into the air. It is preferred that the hydrophilic material comprises anhydrous magnesium carbonate. Additionally, it is preferred that the magnesium carbonate is amorphous. In testing performed by the inventors, it was found that the magnesium carbonate can be formulated to re-release up to 95% of the absorbed water, in exemplary embodiments of the instant invention.

The catalytic material in the catalytic target structure 180 may play a key role in catalyzing the formation of advanced oxidation products within and at the surface of the structure. The catalytic material is preferably titanium dioxide. At least a portion of the titanium dioxide is in anatase crystal form. In exemplary embodiments, almost all of the titanium dioxide is in anatase crystal form, i.e. at least 90%, at least 95%, or at least 99% of the titanium dioxide is in anatase crystal form. In exemplary embodiments, at least a portion of the titanium dioxide is in the form of nanoparticles.

The ceramic matrix provides structural support, and allows for production of a more rigid final material. Preferably, the ceramic matrix comprises cerium oxide and aluminum oxide (Al₂O₃). The cerium oxide acts as a binder with the Al₂O₃. Additionally, the cerium oxide has inherent hydration properties, i.e. it is hydrophilic, and thus further enhances the effect of the MgCO₃ described above. The cerium oxide also has inherent catalytic properties.

In addition, one or more known catalytic enhancers or dopants can optionally be added during the process of forming the wick structure, such that the catalytic enhancer(s) or dopant(s) are integrated into the final wick structure. Known catalytic enhancers and dopants appropriate for inclusion in the catalytic target structure 180 may include, but are not limited to, rhodium, silver, copper, zinc, platinum, nickel, erbium, yttrium, fluorine, sodium, ytterbium, boron, nitrogen, phosphorus, oxygen, thulium, silicon, niobium, sulfur, chromium, cobalt, vanadium, iron, manganese, tungsten, ruthenium, gold, palladium, cadmium, and bismuth, and combinations thereof.

The above-described device and method offers several distinct advantages. In some embodiments, the device incorporates a photocatalytic configuration which initiates a chemical reaction utilizing airborne oxygen and water producing reactive oxygen species including hydrogen peroxide, hydroxyls, hydroperoxyls, singlet oxygen and others as gases. With the exception of hydrogen peroxide these can be short lived compounds which chemically react with gases and particles, as well as surface contaminants.

In some embodiments, the device 100 may include sensors that receive inputs from the environment. These inputs may be used when needed to control the device 100 and subsequently improve the lifetime operation of the device 100 by optimizing the device's 100 functions to the environment.

In some embodiments, the device 100 has reflectors 150 that are positioned perpendicular to the air flow (see, e.g., FIGS. 1 and 2), and parallel to the UV sources 140 and PHI catalytic structure 180. This enables an effective UV output increase to occur, by UV photons being reflected or “pumped” repeatedly within the cell reactor (similar to how a laser diode pump is used to increase laser output). In some embodiments, this can be used to create a much more UV intense field, to both treat the air itself with a higher delivered dose/intensity of germicidal UV (256-275 nm), but also to increase the reactivity and effectiveness of the PHI (photocatalytic reaction surfaces), as higher delivered UV doses to are achieved on the surface, vs. a non-reflector optimized system).

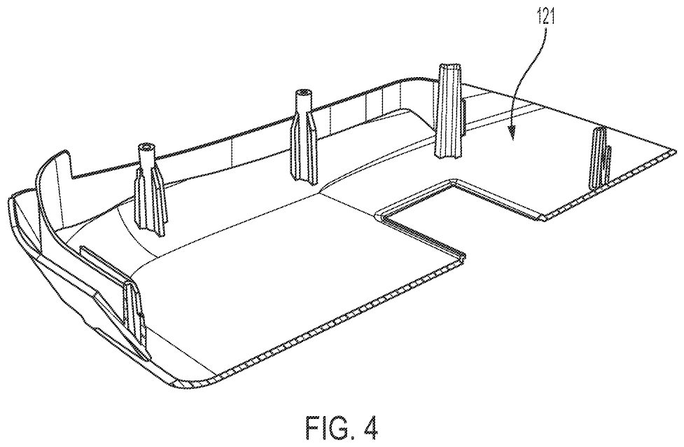

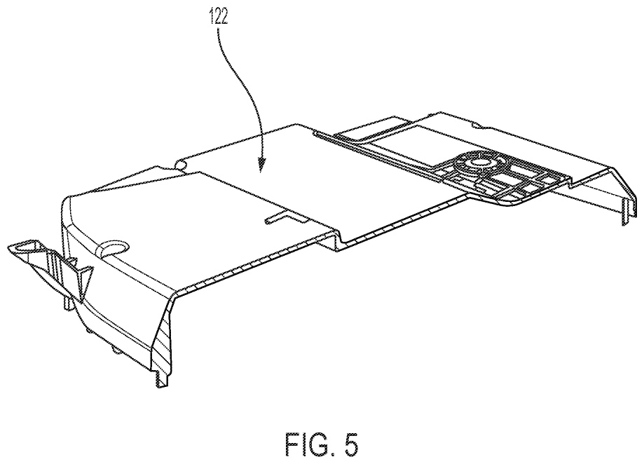

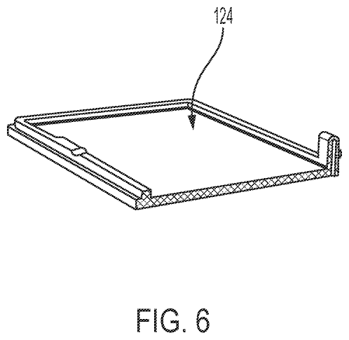

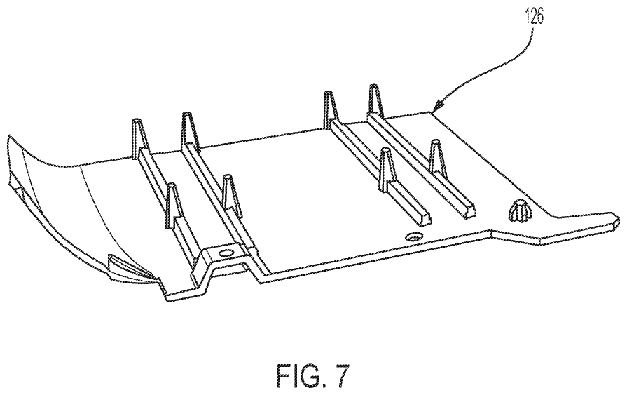

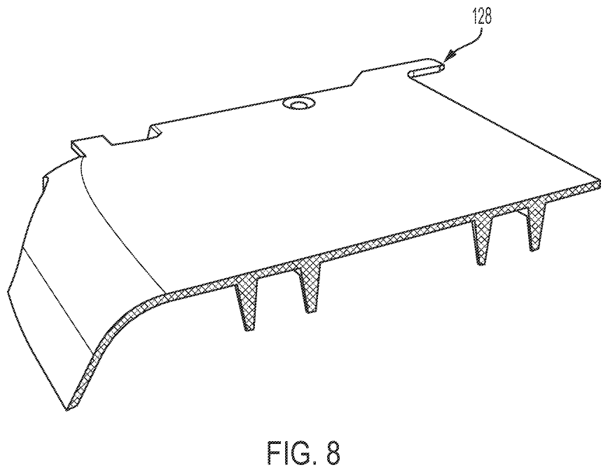

In some embodiments, as shown in FIG. 17, the air purification device 100 may be integrated into a vehicle cabin 200. In some embodiments, the device 100 may be housed within the vehicle cabin's 200 cup holder, forming a cup holder system 300. In some embodiments, the device 100 may be housed within the vehicle cabin's 200 duct, forming a duct system 400. In other embodiments, the device 100 may be housed within a vehicle cabin's 200 air conditioning unit, forming an air-conditioning system 500. Further, in some embodiments, the device 100 may be housed within an independent unit in the vehicle cabin 200, forming an independent system 600. It will be understood that the device 100 may not be housed in only these locations in a vehicle cabin 200. It will further be understood that the device 100 may contain all of the components previously described and shown in FIGS. 1-16. However, it will also be understood that the device 100 may be modified in some embodiments to fit within the vehicle cabin 200.

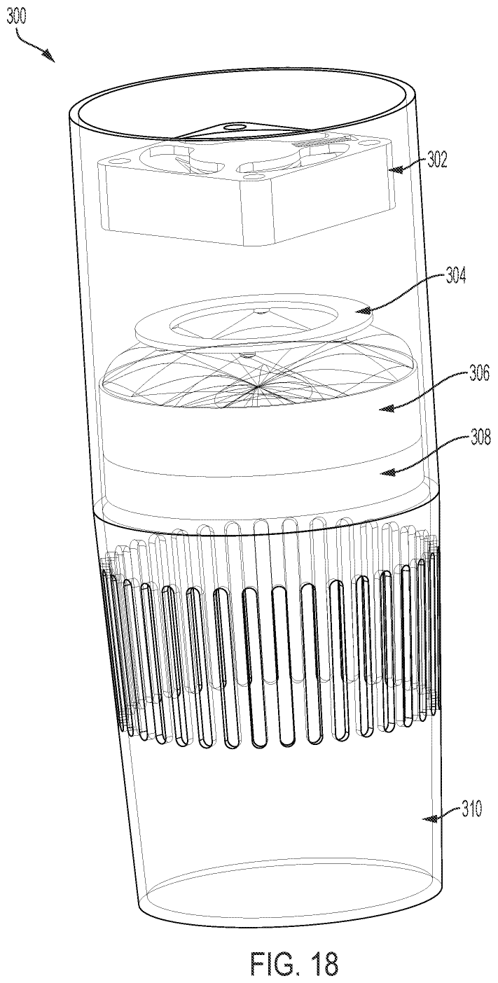

As shown in FIG. 18, in some embodiments, the device 100 is located within a vehicle cabin's 200 cup holder, forming a cup holder purification system 300. In some embodiments, the cup holder system 300 has a fan 302, a UVC-UVA LED ring 304, a catalyst 306, and a filter 308. In some embodiments, the catalyst 308 may be a PHI catalyst as previously disclosed. The system 300 is contained in a case 310. In some embodiments, the filter 308 may be a filter as shown in FIGS. 1, 2, and 14. In some embodiments, the fan 302 may be a fan as shown in FIGS. 1, 2, and 14. In some embodiments, the UVC-UVA LED ring 304 may be an LED module as shown in FIGS. 1, 2, and 10-13. The UVC-UVA LED ring 304 can utilize dual or multi-wavelength UV LEDs.

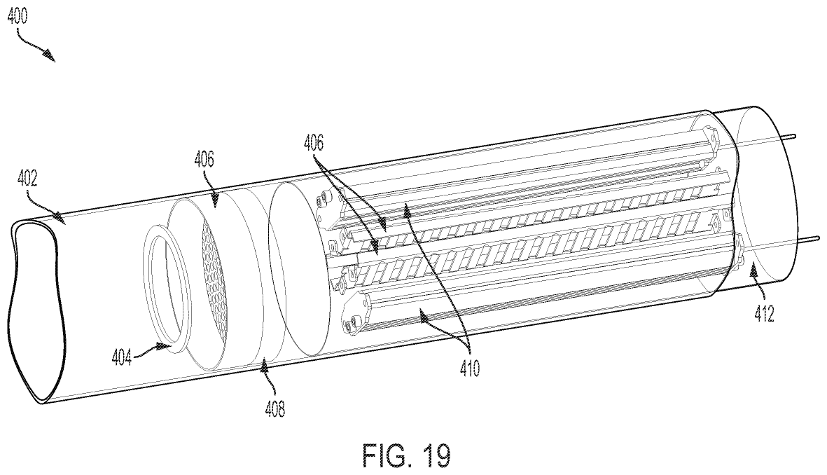

As shown in FIG. 19, in some embodiments, the device 100 is located within a vehicle cabin's 200 air ducts 402, forming a duct purification system 400. In some embodiments, the system 400 contains a LED Ring Board 404, a catalyst 406, a filter 408, a plurality of LED modules 410, and a reflector 412. In some embodiments, the LED Ring Board 404 may be a 365 nm LED Ring Board. The LED Ring Board 404 in some embodiments may be an LED module as shown in FIGS. 1, 2, and 10-13. Further, the LED Ring Board 404 may utilize multiple wavelengths. In some embodiments, the catalyst 408 may be a PHI catalyst as previously disclosed. It will be understood in some embodiments that the filter 408 may be the filters as shown in FIGS. 1, 2, and 14. In other embodiments, the LED modules 410 may range from 265 to 275 nm LED modules. In some embodiments, the reflector 412 may be a PHI reflector. It will additionally be understood that, in some embodiments, the reflector 412 may be the reflector as previously disclosed and shown in FIGS. 1, 2, and FIG. 15. In some embodiments, the reflector 412 may line the circumference of the duct 402; that is, the reflector 412 in some embodiments may be located around the entire inner portion of the duct 402 and below the LED Ring Board 404.

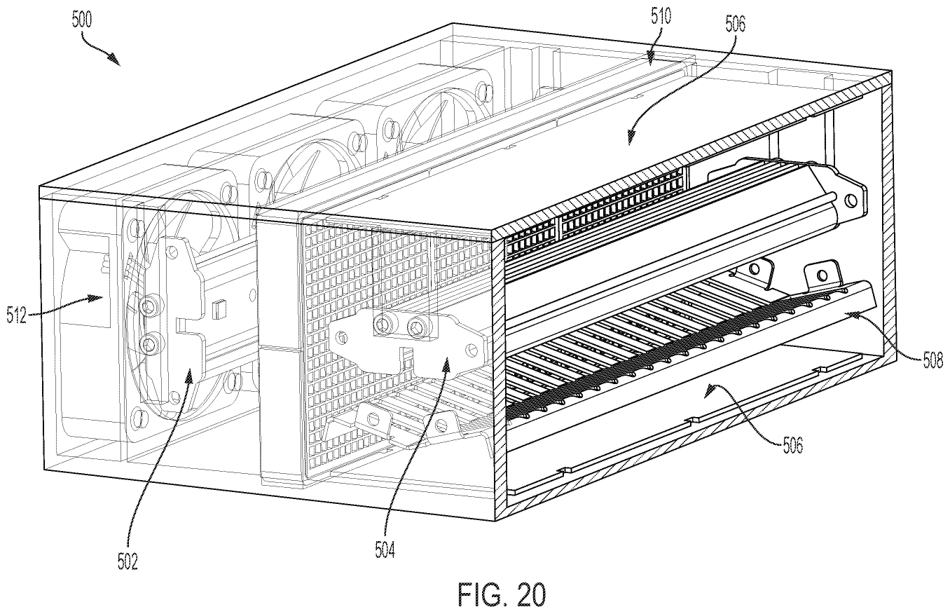

As shown in FIG. 20, in some embodiments, the device 100 is located within a vehicle cabin's 200 air-conditioning vent, forming an air conditioning purification system 500. In some embodiments, the system 500 includes a first LED module 502, a plurality of second LED modules 504, reflectors 506, a catalyst 508, a filter 510, and a series of fans 512. In some embodiments, the first LED module 502 is a 365 nm LED strip, and the plurality of second LED modules 504 may be 265-275 nm LED modules. In some embodiments, the catalyst 508 may be a PHI catalyst as previously disclosed. Further, in some embodiments, the catalyst 508 may be a PHI catalyst. It will additionally be understood that, in some embodiments, the reflectors 506 may be the reflectors as previously disclosed and shown in FIGS. 1, 2, and 15. It will be understood in some embodiments that the filters 510 may be the filters as shown in FIGS. 1, 2, and 14. It will further be understood that the fans 512 may be the fans as shown in FIGS. 1, 2, and 9.

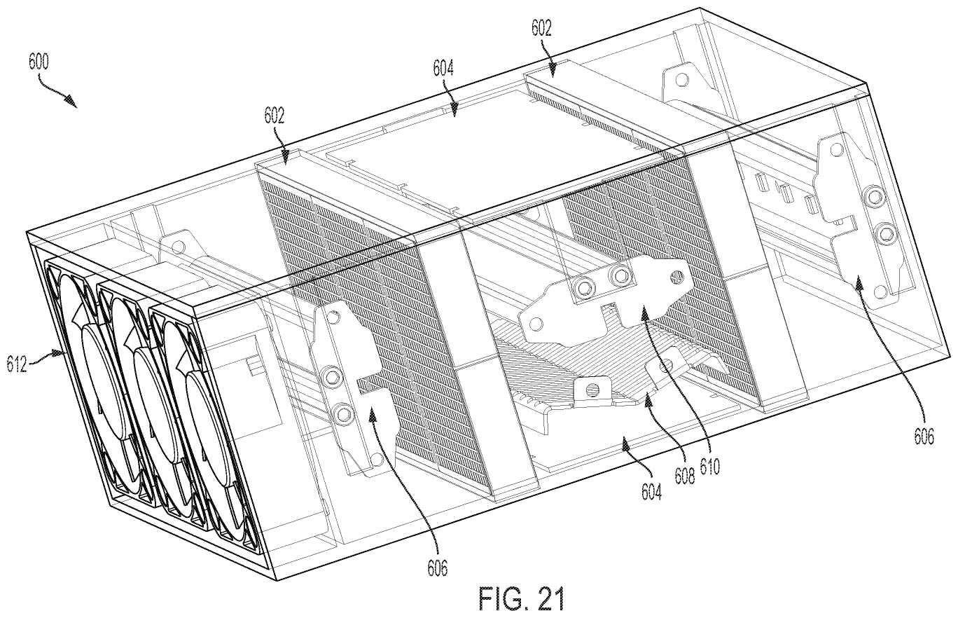

As shown in FIG. 21, in some embodiments, the device 100 is configured for installation in an interior space within the vehicle cabin 200, forming an independent system 600. In some embodiments, the system 600 includes a plurality of filters 602, a plurality of reflectors 604, a first plurality of LED strips 606, a catalyst 608, a second LED strip 610, and a plurality of fans 612. It will be understood in some embodiments that the filters 602 may be the filters as shown in FIGS. 1, 2, and 14. It will additionally be understood that, in some embodiments, the reflectors 604 may be the reflectors as previously disclosed and shown in FIGS. 1, 2, and 15. In some embodiments, the catalyst 608 may be a PHI catalyst as previously disclosed. It will further be understood that the fans 612 may be the fans as shown in FIGS. 1, 2, and 9.

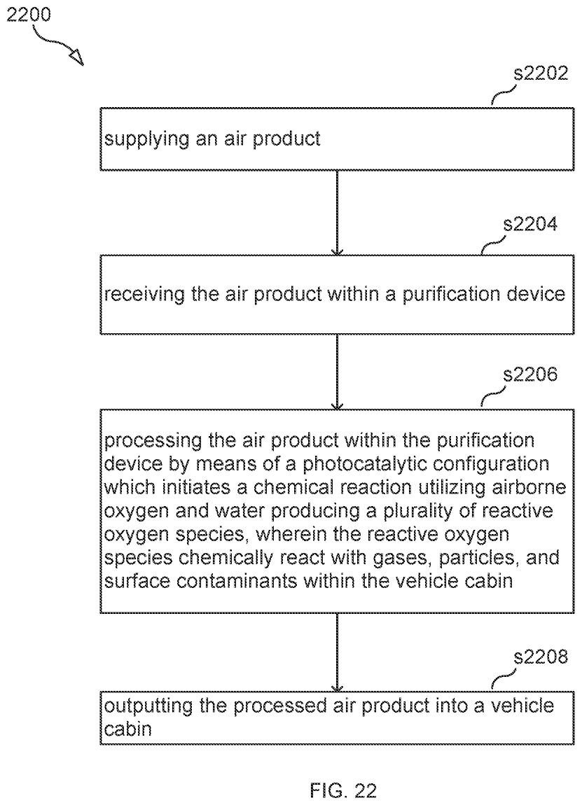

FIG. 22 is a flow chart illustrating a method for purifying a vehicle cabin according to some embodiments. Method 2200 may begin with step s2202.

Step s2202 comprises supplying an air product.

Step s2204 comprises receiving the air product within a purification device.

Step s2206 comprises processing the air product within the purification device by means of a photocatalytic configuration which initiates a chemical reaction utilizing airborne oxygen and water producing a plurality of reactive oxygen species, wherein the reactive oxygen species chemically react with gases, particles, and surface contaminants within the vehicle cabin.

Step s2208 comprises outputting the processed air product into a vehicle cabin.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the disclosed subject matter requires features or combinations of features other than those expressly recited in the embodiments. Accordingly, the scope of this disclosure is intended to include all modifications and variations encompassed within the spirit and scope of the following appended embodiments.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention.

1. A device for purifying a vehicle cabin, the device comprising:
a housing;
a plurality of light emitting diode (LED) modules each containing an LED, wherein the LED modules are positioned at least partially within the housing;
a catalytic target structure, wherein the structure is located below at least one of the LED modules in the plurality of LED modules;
a plurality of reflectors, wherein the reflectors are located below at least one of the LED modules in the plurality of LED modules;
a plurality of fans, wherein the fans are located at least partially within the housing;
a plurality of photocatalyst filters positioned at least partially within the housing,
wherein at least one of the plurality of photocatalyst filters is in parallel with at least one of the LED modules in the plurality of LED modules; and
a control unit located at least partially within the housing, wherein the control unit is operatively connected to the plurality of LED modules.

2. The device according to claim 1, wherein the plurality of LED modules comprises:
a first LED module positioned at least partially within the housing, wherein the first LED module emits ultraviolet light at a first wavelength;
a second LED module positioned at least partially within the housing, wherein the second LED module emits ultraviolet light at a second wavelength;
a third LED module positioned at least partially within the housing, wherein the third LED module emits ultraviolet light at a third wavelength.

3. The device according to claim 2, wherein the first LED module and the third LED module emit ultraviolet light at a wavelength between 300 and 400 nm and the second LED module emits ultraviolet light at a wavelength between 200 and 300 nm.

4. The device according to claim 3, wherein the first LED module and the third LED module emit ultraviolet light at a wavelength of 365 nm and the second LED module emits ultraviolet light at a wavelength of 265-275 nm.

5. The according to claim 2, wherein the catalytic target structure is located below the second LED module.

6. The according to claim 2, wherein the plurality of reflectors comprises:
a first reflector located above the second LED module; and
a second reflector located below the second LED module.

7. The device according to claim 1, wherein at least one of the plurality of reflectors is a flat surface comprising a highly UV reflective material.

8. The device according to claim 7, wherein the reflective material is selected from a group consisting of aluminum, aluminum foil, stainless steel, and polytetrafluoroethylene.

9. The device according to claim 6, wherein the first reflector is located above the second LED module, the catalytic target structure is located below the second LED module and the second reflector is located below the catalytic target structure.

10. The device according to claim 2, wherein the plurality of photocatalyst filters comprises a first photocatalyst filter and a second photocatalyst filter, wherein the first LED module is in parallel with the first photocatalyst filter and the second LED module is in parallel with the second photocatalyst filter.

11. The device according to claim 1, wherein the control unit is configured to control at least one of the plurality of LED modules. the fan series, and the catalytic target structure.

12. The device according to claim 11, wherein the control unit is configured to control ambient conditions within the housing.

13. The device according to claim 12, wherein the ambient conditions are selected from a group consisting of humidity, temperature, selective gases, noise level, and air quality.

14. The device according to claim 1, wherein the plurality of fans are positioned in a series.

15. The device according to claim 1, wherein the device emits zero to near zero ozone.

16. The device according to claim 1, wherein the device is configured such that the device generates 10-70 parts per billion of ROS compounds in the vehicle cabin the device is purifying.

17. The device according to claim 1, wherein at least one of the plurality of photocatalyst filters is in a honeycomb configuration.

18. The device o according to claim 1, wherein at least one of the plurality of photocatalyst filters is composed of a material selected from a group consisting of aluminum oxide, silicon dioxide, magnesium oxide, and titanium oxide.

19. A method for purifying a vehicle cabin, the method comprising:
supplying an air product;
receiving the air product within a purification device;
processing the air product within the purification device by means of a photocatalytic configuration which initiates a chemical reaction utilizing airborne oxygen and water producing a plurality of reactive oxygen species,
wherein the reactive oxygen species chemically react with gases, particles, and surface contaminants within the vehicle cabin; and
outputting the processed air product into a vehicle cabin.

20. The method according to claim 19, wherein the reactive oxygen species is selected from a group consisting of hydrogen peroxide, hydroxyls, hydroperoxyls, and singlet oxygen.

21. A system for purifying a vehicle cabin, the system comprising:
an air supply that supplies an air product;
a purification device configured to receive the air product and output processed air, the device comprising:
a housing;
a plurality of light emitting diode (LED) modules each containing an LED, wherein the LED modules are positioned at least partially within the housing;
a catalytic target structure, wherein the structure is located below at least one of the LED modules in the plurality of LED modules;
a plurality of reflectors, wherein the reflectors are located below at least one of the LED modules in the plurality of LED modules;
a plurality of fans, wherein the fans are located at least partially within the housing;
a plurality of photocatalyst filters positioned at least partially within the housing,
wherein at least one of the plurality of photocatalyst filters is in parallel with at least one of the LED modules in the plurality of LED modules; and
a control unit located at least partially within the housing, wherein the control unit
is operatively connected to the plurality of LED modules; and
a vehicle cabin that receives the processed air output from the purification device.

22. The system according to claim 22, wherein the purification device is disposed in at least one of a vehicle air conditioning system, a vehicle pillar, and a vehicle cabin.
